(19) United States
(12) Patent Application Publication
(10) Pub. No.: US 2018/0143205 A1
(43) Pub. Date: May 24, 2018

(54) BIOMARKERS FOR PREDICTING DEGREE OF WEIGHT LOSS IN FEMALE SUBJECTS

(71) Applicant: NESTEC S.A., CH-1800 Vevery (CH)

(72) Inventors: Irina IRINCHEEVA, Cossonay-Ville (CH); Jorg HAGER, Houtaud (FR); Loic DAYON, Colombier (CH); Ornella COMINETTI, Preverenges (CH)

(21) Appl. No.: 15/570,124

(22) PCT Filed: Apr. 12, 2016
(86) PCT No.: PCT/EP2016/057988

§ 371 Date: Oct. 27, 2017

(30) Foreign Application Priority Data
Apr. 22, 2015 (EP) 15164735.1

(51) Int. Cl. G01N 33/68; G01N 33/92; G01N 33/573; G16H 20/60; A23L 33/00

(52) U.S. Cl. CPC G01N 33/6893; G01N 33/92; G01N 33/573; C12Y 304/21034; G16H 20/60; A23L 33/30; A23V 2002/00; G01N 2333/775; G01N 2333/96455; G01N 2800/52; G01N 2333/81; G01N 2333/811; G01N 2800/044; G01N 2333/4703

(57) ABSTRACT

A method for predicting the degree of weight loss in a female subject attainable by applying one or more dietary interventions to a subject, said method comprising; determining the level of one or more biomarkers in one or more samples obtained from the subject, wherein the biomarkers are selected from gelsolin, apolipoprotein B-100, plasma kallikrein, protein Z-dependent protease inhibitor and plasma serine protease inhibitor.

### FIELD OF INVENTION

The present invention provides a number of biomarkers and biomarker combinations that can be used to determine the gender-specific weight loss trajectory of an individual and further provides methods of optimizing dietary interventions.

### BACKGROUND

Obesity is a chronic metabolic disorder that has reached epidemic proportions in many areas of the world. Obesity is the major risk factor for serious co-morbidities such as type 2 diabetes mellitus, cardiovascular disease, dyslipidaemia and certain types of cancer (World Health Organ Tech Rep Ser. 2000; 894:i-xii, 1-253).

It has long been recognized that low calorie dietary interventions can be very efficient in reducing weight and that this weight loss is generally accompanied by an improvement for the risk of obesity related co-morbidities, in particular type 2 diabetes mellitus (World Health Organ Tech Rep Ser. 2000; 894:i-xii, 1-253). Empirical data suggests that a weight loss of at least 10% of the initial weight results in a considerable decrease in risk for obesity related co-morbidities (World Health Organ Tech Rep Ser. 2000; 894:i-xii, 1-253). However, the capacity to lose weight shows large inter-subject variability.

Some studies (e.g. Ghosh, S. et al., Obesity (Silver Spring), (2011) 19(2):457-463) illustrate that a percentage of the population do not successfully lose weight on a low calorie diet. This leads to an unrealistic expectation of weight loss, which in turn causes non-compliance, drop-outs and generally unsuccessful dietary intervention.

Some studies also demonstrate that there are methods in the art for monitoring weight loss which include monitoring levels of particular biomarkers in plasma (e.g. Lijnen et al., Thromb Res. 2012 January, 129(1): 74-9; Cugno et al., Intern Emerg Med. 2012 June, 7(3): 237-42; and Bladbjerg et al., Br J Nutr. 2010 December, 104(12): 1824-30). However, these methods do not provide a prediction or indication of the degree of weight loss attainable by a particular subject. There is no predictive value in looking at the correlation of biomarker levels with weight loss.

The solution for successful planning and design of dietary interventions, for example low calorie diets, lies in the availability of a method which predicts a weight loss trajectory. Such a method would be useful to assist in modifying a subject's lifestyle, e.g. by a change in diet, and also to stratify subjects into adapted treatment groups according to their biological weight loss capacity.

United States Patent Application US 2011/0124121 discloses a method for predicting weight loss success. The methods disclosed comprises selecting a patient who is undergoing or considering undergoing a weight loss therapy such as gastric banding, measuring one or more hormone responses of the patient to caloric intake and predicting success of a weight loss therapy based on the hormone response. The hormones measured are gastrointestinal hormones such as a pancreatic hormone.

European Patent Application EP 2 420 843 discloses a method for determining the probability that a person will maintain weight loss after an intentional weight loss by determining the level of angiotensin I converting enzyme (ACE) before and after the dietary period.

There is, however, still a need for a method of accurately predicting the degree of weight loss in a subject. Moreover, it is widely known that males and females have different mechanisms of fat storage and metabolisms (Power and Schulkin., Br J Nutr. 2008; 99:931-40; Mittendorfer et al., Obesity (Silver Spring). 2009; 17:1872-7; Menegoni et al., Obesity (Silver Spring). 2009; 17:1951-6), yet these biological sex-specific differences are largely absent from weight loss studies.

Consequently, it was the objective of the present invention to provide biomarkers that can be detected easily and that can facilitate gender-specific prediction of weight loss in a subject. Such biomarkers can be used to predict weight trajectory of a subject prior to a dietary intervention. These biomarkers can be used to optimise dietary intervention and assist in lifestyle modifications.

### SUMMARY OF THE INVENTION

The present invention investigates the level of one or more biomarkers in order to predict the degree of weight loss attainable by applying one or more dietary interventions to a female subject. In particular, the present invention provides gender-specific biomarkers that allow the accurate prediction of weight trajectory of a subject prior to a dietary intervention, for example, a low calorie diet. Accordingly the present invention provides in one aspect a method for predicting the degree of weight loss in a female subject attainable by applying one or more dietary interventions to a subject, said method comprising determining the level of one or more biomarkers in one or more samples obtained from the subject, wherein the biomarkers are selected from gelsolin, apolipoprotein B-100, plasma kallikrein, protein Z-dependent protease inhibitor and plasma serine protease inhibitor.

In one embodiment, the method comprises determining the level of gelsolin and apolipoprotein B-100 in one or more samples.

In one embodiment, the method comprises determining the level of gelsolin and plasma kallikrein in one or more samples.

In one embodiment, the method comprises determining the level of gelsolin and protein Z-dependent protease inhibitor in one or more samples.

In one embodiment, the method comprises determining the level of gelsolin and plasma serine protease inhibitor in one or more samples.

In one embodiment, the method comprises determining the level of apolipoprotein B-100 and plasma kallikrein in one or more samples.

In one embodiment, the method comprises determining the level of apolipoprotein B-100 and protein Z-dependent protease inhibitor in one or more samples.

In one embodiment, the method comprises determining the level of apolipoprotein B-100 and plasma serine protease inhibitor in one or more samples.

In one embodiment, the method comprises determining the level of plasma kallikrein and protein Z-dependent protease inhibitor in one or more samples.

In one embodiment, the method comprises determining the level of plasma kallikrein and plasma serine protease inhibitor in one or more samples.

In one embodiment, the method comprises determining the level of protein Z-dependent protease inhibitor and plasma serine protease inhibitor in one or more samples.

In one embodiment, the method comprises determining the level of gelsolin, apolipoprotein B-100 and plasma kallikrein in one or more samples.

In one embodiment, the method comprises determining the level of gelsolin, apolipoprotein B-100 and protein Z-dependent protease inhibitor in one or more samples.

In one embodiment, the method comprises determining the level of gelsolin, apolipoprotein B-100 and plasma serine protease inhibitor in one or more samples.

In one embodiment, the method comprises determining the level of gelsolin, plasma kallikrein and protein Z-dependent protease inhibitor in one or more samples.

In one embodiment, the method comprises determining the level of gelsolin, plasma kallikrein and plasma serine protease inhibitor in one or more samples.

In one embodiment, the method comprises determining the level of gelsolin, protein Z-dependent protease inhibitor and plasma serine protease inhibitor in one or more samples.

In one embodiment, the method comprises determining the level of apolipoprotein B-100, plasma kallikrein and protein Z-dependent protease inhibitor in one or more samples.

In one embodiment, the method comprises determining the level of apolipoprotein B-100, plasma kallikrein and plasma serine protease inhibitor in one or more samples.

In one embodiment, the method comprises determining the level of apolipoprotein B-100, protein Z-dependent protease inhibitor and plasma serine protease inhibitor in one or more samples.

In one embodiment, the method comprises determining the level of plasma kallikrein, protein Z-dependent protease inhibitor and plasma serine protease inhibitor in one or more samples.

In one embodiment, the method comprises determining the level of gelsolin, apolipoprotein B-100, plasma kallikrein and protein Z-dependent protease inhibitor and in one or more samples.

In one embodiment, the method comprises determining the level of gelsolin, apolipoprotein B-100, plasma kallikrein and plasma serine protease inhibitor in one or more samples.

In one embodiment, the method comprises determining the level of gelsolin, apolipoprotein B-100, protein Z-dependent protease inhibitor and plasma serine protease inhibitor in one or more samples.

In one embodiment, the method comprises determining the level of gelsolin, plasma kallikrein, protein Z-dependent protease inhibitor and plasma serine protease inhibitorin one or more samples.

In one embodiment, the method comprises determining the level of apolipoprotein B-100, plasma kallikrein, protein Z-dependent protease inhibitor and plasma serine protease inhibitor in one or more samples.

In one embodiment, the method comprises determining the level of gelsolin, apolipoprotein B-100, plasma kallikrein, protein Z-dependent protease inhibitor and plasma serine protease inhibitor in one or more samples.

In one embodiment, the one or more samples are derived from blood, e.g. a blood plasma sample.

The level of the one or more biomarkers may be compared to a reference value, wherein the comparison is indicative of the predicted degree of weight loss attainable by the subject. The reference value may be based on a value (e.g. an average) of the one or more biomarkers in a population of subjects who have previously undergone the dietary intervention.

In one embodiment, a level of gelsolin is determined, and an increase in the level of gelsolin in the sample relative to a reference value is indicative of a greater degree of weight loss in a subject.

In one embodiment, a level of apolipoprotein B-100 is determined, and a decrease in the level of apolipoprotein B-100 in the sample relative to a reference value is indicative of a greater degree of weight loss in a subject.

In one embodiment, a level of plasma kallikrein is determined, and an increase in the level of plasma kallikrein in the sample relative to a reference value is indicative of a greater degree of weight loss in a subject.

In one embodiment, a level of protein Z-dependent protease inhibitor is determined, and an increase in the level of protein Z-dependent protease inhibitor in the sample relative to a reference value is indicative of a greater degree of weight loss in a subject.

In one embodiment, a level of plasma serine protease inhibitor is determined, and a decrease in the level of plasma serine protease inhibitor in the sample relative to a reference value is indicative of a greater degree of weight loss in a subject.

In another embodiment, levels of each of gelsolin, apolipoprotein B-100, plasma kallikrein, protein Z-dependent protease inhibitor, and plasma serine protease inhibitor are determined, and decreased levels of apolipoprotein B-100 and plasma serine protease inhibitor and increased levels gelsolin, plasma kallikrein and protein Z-dependent protease inhibitor in the sample is indicative of a greater degree of weight loss in the subject.

Preferably the dietary intervention is a low calorie diet. In one embodiment, the low calorie diet comprises a calorie intake of about 600 to about 1200 kcal/day. The low calorie diet may comprise administration of at least one diet product. Preferably the diet product is Optifast® or Modifast®. The low calorie diet may also comprise administration of up to, for example, about 400 g vegetables/day.

In one embodiment, the diet may comprise a product such as Optifast® or Modifast®. This may be supplemented with three portions of non-starchy vegetables such that the total energy intake is about 2.5 MJ (600 kcal/day). This may be further supplemented with at least 2 L of water or other energy free beverages per day.

In another embodiment, the diet may comprise, for example, a composition which is 46.4% carbohydrate, 32.5% protein and 20.1% fat, vitamins, minerals and trace elements; 2.1 MJ per day (510 kcal/day); This may be supplemented with three portions of non-starchy vegetables such that the total energy intake is about 2.5 MJ (600 kcal/day). This may be further supplemented with at least 2 L of water or other energy free beverages per day.

In one embodiment, the low calorie diet has a duration of up to 12 weeks, e.g. 6 to 12 weeks.

In one embodiment, the method further comprises combining the level of the one or more biomarkers with one or more anthropometric measures and/or lifestyle characteristics of the subject. In one embodiment the anthropometric measure is selected from the group consisting of weight, height, age and body mass index, and the lifestyle characteristic is whether the subject is a smoker or a non-smoker.

In one embodiment, the method further comprises combining the level of the one or more biomarkers with one or more anthropometric measures which include age and body mass index.

In one embodiment, the degree of weight loss is represented by the body mass index (BMI) that a subject is predicted to attain by applying the dietary intervention. This may be termed BMI2 and be calculated using formula (1):

bmi2ᵢ=c1*bmi1ᵢ+c2*ageᵢ−c3*gelsolinᵢ−c4*plasma kallikreinᵢ−c5*protein Z-dependent protease inhibitorᵢ+c6*apolipoprotein B-100ᵢ+c7*plasma serine protease inhibitorᵢ

wherein BMI1 is the subject's body mass index before the dietary intervention and BMI2 is the subject's predicted body mass index after the dietary intervention; and wherein c1, c2, c3, c4, c5, c6 and c7 are positive integers.

According to a further aspect, the present invention provides a method for optimizing one or more dietary interventions for a female subject comprising predicting the degree of weight loss attainable by the subject according to a method as defined herein and applying the dietary intervention to the subject.

In a further aspect of the present invention there is provided a method for predicting the body mass index that a female subject would be expected to attain from a dietary intervention (BMI2), wherein the method comprises determining the level of gelsolin, apolipoprotein B-100, plasma kallikrein, protein Z-dependent protease inhibitor and plasma serine protease inhibitor in one or more samples obtained from the subject, and predicting BMI2 using formula (1) as described hereinabove.

In a further aspect of the present invention there is provided a method for selecting a modification of lifestyle of a female subject, the method comprising (a) performing a method as defined herein, and (b) selecting a suitable modification in lifestyle based upon the degree of weight loss predicted.

In one embodiment, the modification of lifestyle comprises a dietary intervention. The dietary intervention may comprise administering at least one diet product to the subject. For example, the dietary intervention may be a low calorie diet. A low calorie diet may comprise a decreased consumption of fat and/or an increase in consumption of low fat foods. By way of example only, low fat foods may include wholemeal flour and bread, porridge oats, high-fibre breakfast cereals, wholegrain rice and pasta, vegetables and fruit, dried beans and lentils, baked potatoes, dried fruit, walnuts, white fish, herring, mackerel, sardines, kippers, pilchards, salmon and lean white meat.

In a further aspect of the present invention there is provided a diet product for use as part of a low calorie diet for weight loss, wherein the diet product is administered to a female subject that is predicted to attain a degree of weight loss by the method described herein.

In one aspect, the diet product may comprise a product such as Optifast® or Modifast®. This may be supplemented with three portions of non-starchy vegetables such that the total energy intake is about 2.5 MJ (600 kcal/day). This may be further supplemented with at least 2 L of water or other energy free beverages per day.

In another aspect, the diet product may comprise, for example, a composition which is 46.4% carbohydrate, 32.5% protein and 20.1% with fat, vitamins, minerals and trace elements; 2.1 MJ per day (510 kcal/day); This may be supplemented with three portions of non-starchy vegetables such that the total energy intake is about 2.5 MJ (600 kcal/day). This may be further supplemented with at least 2 L of water or other energy free beverages per day.

In a further aspect of the present invention there is provided a diet product for use in treating obesity or an obesity-related disorder, wherein the diet product is administered to a female subject that is predicted to attain a degree of weight loss by the methods defined herein

In a further aspect of the present invention there is provided a diet product for use in treating obesity or an obesity-related disorder, wherein the diet product is administered to a female subject that is predicted to attain a degree of weight loss by the methods defined herein.

In a further aspect of the present invention, there is provided the use of a diet product in a low calorie diet for weight loss wherein the diet product is administered to a female subject that is predicted to attain a degree of weight loss by the methods defined herein.

In a further aspect of the present invention, there is provided a computer program product comprising computer implementable instructions for causing a programmable computer to predict the degree of weight loss attainable by a female subject according to the methods described herein.

In a further aspect of the present invention, there is provided a computer program product comprising computer implementable instructions for causing a programmable computer to predict the degree of weight loss given the levels of one or more biomarkers from the user, wherein the biomarkers are selected from gelsolin, apolipoprotein B-100, plasma kallikrein, protein Z-dependent protease inhibitor and plasma serine protease inhibitor.

In a further aspect of the present invention, there is provided a kit for predicting the degree of weight loss attainable by a female subject following a dietary intervention, wherein said kit comprises two or more antibodies selected from the group consisting of an antibody specific for gelsolin, an antibody specific for apolipoprotein B-100, an antibody specific for plasma kallikrein, an antibody specific for protein Z-dependent protease inhibitor and an antibody specific for plasma serine protease inhibitor.

In one embodiment, the kit further comprises an antibody specific for gelsolin, an antibody specific for apolipoprotein B-100, an antibody specific plasma kallikrein, an antibody specific for protein Z-dependent protease inhibitor, and an antibody specific for plasma serine protease inhibitor.

### DETAILED DESCRIPTION OF THE INVENTION

### Predicting the Degree of Weight Loss

The present invention relates in one aspect to a method of predicting the degree of weight loss attainable by applying one or more dietary interventions to a female subject. In particular embodiments, the method may be used to make an informed prediction of the subject's capacity to lose weight, and select or adjust one or more dietary interventions accordingly. For example, where the dietary intervention is a low calorie diet, the method could be used to select the appropriate diet for the subject or to adjust the daily calorie intake or duration of a particular diet to affect the degree of weight loss, or to increase compliance to the low calorie diet by setting realistic expectations for the subject. The method may also be used to assist in modifying the lifestyle of a subject.

The method provides a skilled person with a useful tool for assessing which subjects will most likely benefit from a particular dietary intervention, e.g. a low calorie diet. The present method therefore enables dietary interventions such as a low calorie diet and modifications in lifestyle to be optimised.

Weight loss as defined herein may refer to a reduction in parameters such as weight (e.g. in kilograms), body mass index (e.g. kgm⁻²), or waist circumference (e.g. in centimetres), or waist-hip ratio (e.g. in centimetres). Weight loss may be calculated by subtracting the value of one or more of the aforementioned parameters at the end of the dietary intervention from the value of said parameter at the onset of the dietary intervention. Preferably, the degree of weight loss is represented by the body mass index that a subject is predicted to attain by applying the dietary intervention.

The degree of weight loss may be expressed as a percentage of a subject's body weight (e.g. in kilograms) or body mass index (kgm⁻²). For example, a subject may be predicted to lose at least 10% of their initial body weight, at least 8% of their initial body weight, or at least 5% of their initial body weight. By way of example only, a subject may be predicted to lose between 5 and 10% of their initial body weight.

In one embodiment, the percentage may be associated with an obesity-related disorder. For example, a degree of weight loss of at least 10% of initial body weight results in a considerable decrease in risk for obesity related co-morbidities.

Based on the degree of weight loss predicted using the methods defined herein, subjects may be stratified into one or more groups or categories. For example, subjects may be stratified according to whether or not they are predicted to lose a significant amount of weight.

Subject

Preferably the subject is a mammal, preferably a human. The subject may alternatively be a non-human mammal, including for example, a horse, cow, sheep or pig. In one embodiment, the subject is a companion animal such as a dog or a cat. According to the present invention, the subject is female.

Sample

The present invention comprises a step of determining the level of one or more biomarkers in one or more samples obtained from a subject.

Preferably the sample is derived from blood. The sample may contain a blood fraction or may be wholly blood. The sample preferably comprises blood plasma or serum, most preferably blood plasma. Techniques for collecting samples from a subject are well known in the art.

Dietary Intervention

By the term “dietary intervention” is meant an external factor applied to a subject which causes a change in the subject's diet. In one embodiment, the dietary intervention is a low calorie diet.

Preferably the low calorie diet comprises a calorie intake of about 600 to about 1500 kcal/day, more preferably about 600 to about 1200 kcal/day, most preferably about 800 kcal/day. In one embodiment, the low calorie diet may comprise a predetermined amount (in grams) of vegetables per day, preferably up to about 400 g vegetables/day, e.g. about 200 g vegetables/day.

The low calorie diet may comprise administration of at least one diet product. The diet product may be a meal replacement product or a supplement product which may e.g. suppress the subject's appetite. The diet product can include food products, drinks, pet food products, food supplements, nutraceuticals, food additives or nutritional formulas.

In one embodiment, the diet may comprise a product such as Optifast® or Modifast®. This may be supplemented with three portions of non-starchy vegetables such that the total energy intake is about 2.5 MJ (600 kcal/day). This may be further supplemented with at least 2 L of water or other energy free beverages per day.

In another embodiment, the diet may comprise, for example, a composition which is 46.4% carbohydrate, 32.5% protein and 20.1% with fat, vitamins, minerals and trace elements; 2.1 MJ per day (510 kcal/day); This may be supplemented with three portions of non-starchy vegetables such that the total energy intake is about 2.5 MJ (600 kcal/day). This may be further supplemented with at least 2 L of water or other energy free beverages per day.

In one embodiment, the low calorie diet has a duration of up to 12 weeks. Preferably the low calorie diet has a duration of between 6 and 12 weeks, preferably between 8 and 10 weeks, e.g. 8 weeks.

Determining the Level of One or More Biomarkers in the Sample

In one embodiment, the level of one or more biomarkers is determined prior to the dietary intervention. In another embodiment, the level of one or more biomarkers is determined prior to, and after the dietary intervention. The biomarker level may also be determined at predetermined times throughout the dietary intervention. These predetermined times may be periodic throughout the dietary intervention, e.g. every day or three days, or may depend on the subject being tested, the type of sample being analysed and/or the degree of weight loss which is predicted to be attained.

When obtained prior to the dietary intervention, the biomarker level may be termed the “fasting level”. When obtained after the dietary intervention, the biomarker level may be termed the “calorie intake level”. For example, the biomarker level may be determined at fasting, or at fasting and after calorie intake. Most preferably the fasting level of each biomarker is determined.

The level of the individual biomarker species in the sample may be measured or determined by any suitable method known in the art. For example, mass spectrometry (MS), aptamer or antibody detection methods, e.g. enzyme-linked immunoabsorbent assay (ELISA) may be used. Other spectroscopic methods, chromatographic methods, labelling techniques, or quantitative chemical methods may also be used.

In one embodiment, the level of one or more biomarkers may be determined by staining the sample with a reagent that labels one or more of the biomarkers. “Staining” is typically a histological method which renders the biomarker detectable by microscopic techniques such as those using visible or fluorescent light. Preferably the biomarker is detected in the sample by immunohistochemistry (IHC). In IHC, the biomarker may be detected by an antibody which binds specifically to one or more of the biomarkers. Suitable antibodies are known or may be generated using known techniques. Suitable test methods for detecting antibody levels include, but are not limited to, an immunoassay such as an enzyme-linked immunosorbent assay, radioimmunoassay, Western blotting and immunoprecipitation.

The antibody may be a monoclonal antibody, polyclonal antibody, multispecific antibody (e.g., bispecific antibody), or fragment thereof provided that it specifically binds to the biomarker being detected. Antibodies may be obtained by standard techniques comprising immunizing an animal with a target antigen and isolating the antibody from serum. Monoclonal antibodies may be made by the hybridoma method first described by Kohler et al., Nature 256:495 (1975), or may be made by recombinant DNA methods (see, e.g., U.S. Pat. No. 4,816,567). The monoclonal antibodies may also be isolated from phage antibody libraries using the techniques described in Clackson et al., Nature 352:624-628 (1991) and Marks et al., J. Mol. Biol. 222:581-597 (1991), for example. The antibody may also be a chimeric or humanized antibody. Antibodies are discussed further below.

Two general methods of IHC are available; direct and indirect assays. According to the first assay, binding of antibody to the target antigen is determined directly. This direct assay uses a labelled reagent, such as a fluorescent tag or an enzyme-labelled primary antibody, which can be visualized without further antibody interaction.

In a typical indirect assay, unconjugated primary antibody binds to the antigen and then a labelled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC may be labelled with a detectable moiety. Numerous labels are available, including radioisotopes, colloidal gold particles, fluorescent labels and various enzyme-substrate labels. Fluorescent labels include, but are not limited to, rare earth chelates (europium chelates), Texas Red, rhodamine, fluorescein, dansyl, Lissamine, umbelliferone, phycocrytherin and phycocyanin, and/or derivatives of any one or more of the above. The fluorescent labels can be conjugated to the antibody using known techniques.

Various enzyme-substrate labels are available, e.g. as disclosed in U.S. Pat. No. 4,275,149. The enzyme generally catalyses a chemical alteration of the chromogenic substrate that can be detected microscopically, e.g. under visible light. For example, the enzyme may catalyse a colour change in a substrate, or may alter the fluorescence or chemiluminescence of the substrate. Examples of enzymatic labels include luciferases (e.g. firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are well known.

Typically the method comprises a step of detecting stained regions within the image. Pixels in the image corresponding to staining associated with the biomarker may be identified by colour transformation methods, for instance as disclosed in U.S. Pat. No. 6,553,135 and U.S. Pat. No. 6,404,916. In such methods, stained objects of interest may be identified by recognising the distinctive colour associated with the stain. The method may comprise transforming pixels of the image to a different colour space, and applying a threshold value to suppress background staining. For instance, a ratio of two of the RGB signal values may be formed to provide a means for discriminating colour information. A particular stain may be discriminated from background by the presence of a minimum value for a particular signal ratio. For instance pixels corresponding to a predominantly red stain may be identified by a ratio of red divided by blue (R/B) which is greater than a minimum value.

Kong et al., Am J Clin Nutr, 2013 December; 98(6):1385-94 describes the use of the avidin-biotin-peroxidase method and two independent investigators counting the number of positively stained cells.

Aptamer based detection methods may be employed by the present invention. Aptamers that specifically recognize the biomarker may be synthesized using standard nucleic acid synthesis techniques or selected from a large random sequence pool, for example using the Systematic Evolution of Ligands by Exponential Enrichment (SELEX) technique.

Aptamers can be single strand DNA or RNA sequences that fold in a unique 3D structure having a combination of stems, loops, quadruplexes, pseudoknots, bulges, or hairpins. The molecular recognition of aptamers results from intermolecular interactions such as the stacking of aromatic rings, electrostatic and van der Waals interactions, or hydrogen bonding with a target compound. In addition, the specific interaction between an aptamer and its target is complemented through an induced fit mechanism, which requires the aptamer to adopt a unique folded structure to its target. Aptamers can be modified to be linked with labeling molecules such as dyes, or immobilized on the surface of beads or substrates for different applications.

Aptamers may be paired with nanotechnology, microarray, microfluidics, mass spectrometry and other technologies for quantification in a given sample.

In one embodiment, the biomarker level is compared with a reference value. In which case, the biomarker level in the sample and the reference value are determined using the same analytical method.

Gelsolin

Gelsolin is a calcium-regulated, actin-modulating protein that binds to the plus (or barbed) ends of actin monomers or filaments, preventing monomer exchange (end-blocking or capping). It can promote the assembly of monomers into filaments (nucleation) as well as sever filaments already formed.

Intracellular and extracellular isoforms of gelsolin are known. Extracellular gelsolin (isoform 1) is detectable in plasma and is a member of the extracellular actin scavenger system (Lee & Galbraith; N Eng J Med; 1992; 326; 1335-41). Cell death and tissue injury causes actin to be released to the circulation, the extracellular actin scavenger system depolymerises and removes this actin from the circulation. Gelsolin severs assembled actin filaments and caps the fast growing barbed ends of a free or newly severed filament.

Methods for determining the level of gelsolin are known in the art. For example, Lee et al. describe an in vitro functional assay for measuring levels of plasma gelsolin (Crit. Care. Med.; 2007; 35:849-855). The assay is based on the principle that calcium-activated plasma gelsolin binds pyrene-labeled actin monomers to form a nucleus from which actin polymerizes in the pointed (slowest-growing) end direction. Polymerization rate in each sample is converted to plasma gelsolin concentration by use of a standard curve of recombinant human plasma gelsolin.

Pan et al. describe the use of a commercial enzyme-linked immunosorbent assay (CoTimes, Beijing, China) for determining plasma gelsolin levels (Critical Care; 2013, 17:R149).

An example human plasma gelsolin protein is the human gelsolin protein having the UniProtKB accession number P06396-1. This exemplified sequence is 782 amino acids in length of which amino acids 1 to 27 form a leader sequence.

Apolipoprotein B-100

Apolipoprotein B-100 (ApoB100) is synthesized exclusively by the liver. It is a major protein constituent of LDL and VLDL and functions as a recognition signal for the cellular binding and internalization of LDL particles by the apoB/E receptor.

ApoB100 is encoded by the APOB gene, which encodes two isoforms, ApoB48 and ApoB100. Apo B-48 is generated when a stop codon (UAA) at residue 2153 is created by RNA editing. There appears to be a trans-acting tissue-specific splicing gene that determines which iso form is ultimately produced. As a result of the RNA editing, ApoB48 and ApoB100 share a common N-terminal sequence, but ApoB48 lacks ApoB100's C-terminal LDL receptor binding region.

Methods for determining the level of ApoB100 in a sample are known in the art. For example, Hermans et al. describe that ApoB100 was measured with immunonephelometry on BNII Analyzer (Siemens Healthcare Products GmbH, Marburg, Germany) (Cardiovascular Diabetology 2013, 12:39); whilst Shidfar et al. describe the determination of serum levels of ApoB100 levels using immunoturbidimetry with a Cobas MIRA analyser (Med J Islam Repub Iran. 2014 Sep. 20; 28:100).

An example human ApoB100 is the human ApoB100 having the UniProtKB accession number P04114. This exemplified sequence is 4563 amino acids in length, of which amino acids 1 to 27 form a leader sequence.

Plasma Kallikrein

Kallikreins are a subgroup of serine proteases. In humans, plasma kallikrein (KLKB1) has no known homologue, while tissue kallikrein-related peptidases (KLKs) encode a family of fifteen closely related serine proteases.

Plasma kallirein cleaves Lys-Arg and Arg-Ser bonds. It is synthesised as an inactive precursor, prekallikrein, which must undergo proteolytic processing to become activated. It activates, in a reciprocal reaction, factor XII after its binding to a negatively charged surface. It also releases bradykinin from HMW kininogen and may also play a role in the renin-angiotensin system by converting prorenin into renin.

Methods of determining the level of plasma kallikrein in a sample are known in the art. Levels of plasma kallikrein may be measured by determining plasma kallikrein enzyme activity levels in a sample and comparing these to enzyme activities of known amounts of plasma kallikrein.

For example Jaffa et al. describe the use of an assay for plasma kallikrein which involves determining the hydrolysis of the chromogenic substrate H-D-Pro-Phe-Arg-paranitroanilide (Diabetes; 2003; 52(5); 1215-1221). Levels of plasma kallikrein may be expressed as enzyme units/ml.

An example human plasma kallikrein protein is the human plasma prekallikrein protein having the UniProtKB accession number P03952. This exemplified sequence is 638 amino acids in length, of which amino acids 1 to 19 form a leader sequence.

Protein Z-Dependent Protease Inhibitor

Protein Z-dependent protease inhibitor (also known as Serpin 10) is encoded by the SERPINA10 gene. It inhibits the activity of the coagulation protease factor Xa in the presence of “protein Z, vitamin K-dependent plasma glycoprotein”, calcium and phospholipids and also inhibits factor XIa in the absence of cofactors.

Methods for determining the level of protein Z-dependent protease inhibitor in a sample are known in the art. For example, Kim et al. describe the use of an enzyme-linked immunosorbent assay to determine protein Z-dependent protease inhibitor levels in plasma samples (Journal of Gastroenterology and Hepatology; 2015; 30; 4:784-793). Al-Shanqeeti et al. also describe the use of an enzyme-linked immunosorbent assay to determine protein Z-dependent protease inhibitor levels in plasma samples (Thrombosis and Haemostasis; 2005; 93:3; 399-623).

An example human protein Z-dependent protease inhibitor is the human Protein Z-dependent protease inhibitor having the UniProtKB accession number Q9UK55. This exemplified sequence is 444 amino acids in length, of which amino acids 1 to 21 form a leader sequence.

Plasma Serine Protease Inhibitor

Plasma serine protease inhibitor is a heparin-dependent protease inhibitor present in body fluids and secretions. It is also known as Protein C inhibitor (PCI) and is encoded by the SERPINA5 gene.

It inactivates serine proteases by binding irreversibly to their serine activation site and plays hemostatic roles in the blood plasma (Suzuki et al.; 1984; J. Biochem.; 95:187-195). It acts as a procoagulant and proinflammatory factor by inhibiting the anticoagulant activated protein C factor as well as the generation of activated protein C factor by the thrombin/thrombomodulin complex (Steif et al.; Biol. Chem; 1987; 368: 1427-1433).

Methods for determining the level of protein Z-dependent protease inhibitor in a sample are known in the art. For example, Laurell et al. describe the use of an enzyme-linked immunosorbent assay to determine plasma serine protease levels in biological samples (J Clin Invest. 1992; 89(4):1094-1101).

An example human plasma serine protease inhibitor is the human plasma serine protease inhibitor having the UniProtKB accession number P05154. This exemplified sequence is 406 amino acids in length, of which amino acids 1 to 19 form a leader sequence.

Combinations of Biomarkers

Whilst individual biomarkers may have predictive value in the methods of the present invention, the quality and/or the predictive power of the methods may be improved by combining values from multiple biomarkers.

Thus the method of the present invention may involve determining the level of at least two, at least three, at least four or all five of the biomarkers from those defined herein. The method may comprise determining the level of any combination of biomarkers as defined herein.

A method comprising detecting a combination of biomarkers including gelsolin, apolipoprotein B-100, plasma kallikrein, protein Z-dependent protease inhibitor and plasma serine protease inhibitor is particularly preferred.

In a particularly preferred embodiment, the method comprises determining the level of each of gelsolin, apolipoprotein B-100, plasma kallikrein, protein Z-dependent protease inhibitor, and plasma serine protease inhibitor, where decreased levels of apolipoprotein B-100 and plasma serine protease inhibitor and increased levels gelsolin, plasma kallikrein and protein Z-dependent protease inhibitor in the sample is indicative of a greater degree of weight loss in the subject.

Comparison to a Reference or Control

The present method may further comprise a step of comparing the level of the individual biomarkers in the test sample to one or more reference or control values. The reference value may be associated with a pre-defined ability of a subject to lose weight following dietary intervention. In some embodiments, the reference value is a value obtained previously for a subject or group of subjects following a certain dietary intervention. The reference value may be based on an average level, e.g. a mean or median level, from a group of subjects following the dietary intervention.

Combining the Biomarker Levels with Anthropometric Measures and/or Lifestyle Characteristics

In one embodiment, the present method further comprises combining the level of the one or more biomarkers with one or more anthropometric measures and/or lifestyle characteristics of the subject. By combining this information, an improved predictive model is provided for the degree of weight loss attainable by a subject.

As is known in the art, an anthropometric measure is a measurement of a subject. In one embodiment, the anthropometric measure is selected from the group consisting of age (in years), weight (in kilograms), height (in centimetres), and body mass index (in kgm⁻²). Other anthropometric measures will also be known to the skilled person in the art.

By the term “lifestyle characteristic” is meant any lifestyle choice made by a subject, this includes all dietary intake data, activity measures or data from questionnaires of lifestyle, motivation or preferences. In one embodiment, the lifestyle characteristic is whether the subject is a smoker or a non-smoker. This is also referred to herein as the smoking status of the subject.

In a preferred embodiment, levels of gelsolin, apolipoprotein B-100, plasma kallikrein, protein Z-dependent protease inhibitor and plasma serine protease inhibitor are determined for a sample from the subject and these levels are combined with the age and body mass index of the subject in order to predict the weight loss attainable by the subject. Preferably the degree of weight loss is represented by the body mass index that a subject is predicted to attain by applying the dietary intervention.

In one embodiment, the predicted body mass index (BMI2) is generally represented by formula (1):

bmi2ᵢ=c1*bmi1ᵢ+c2*ageᵢ−c3*gelsolinᵢ−c4*plasma kallikreinᵢ−c5*protein Z-dependent protease inhibitorᵢ+c6*apolipoprotein B-100ᵢ+c7*plasma serine protease inhibitorᵢ;

wherein BMI1 is the subject's body mass index before the dietary intervention and BMI2 is the subject's predicted body mass index after the dietary intervention; and wherein c1, c2, c3, c4, c5, c6 and c7 are positive integers.

The values of c1 to c7 typically depend on 1) the measurement units of all the variables in the model; and 2) provenance (ethnic background) of the considered subject. Each of the coefficients c1 to c7 can be readily determined for particular subject cohorts. As would be understood by the skilled person, a dietary intervention, for example a low calorie diet, may be applied to a subject cohort of interest, the levels of the biomarkers as defined herein may be determined and routine statistical methods may then be used in order to arrive at the values of c1 to c7. Such routine statistical methods may include multiple linear regression with calibration by bootstrap. It is possible to obtain the same estimates with generalized linear or additive models or any other regression-related model with various estimation algorithms, for example, elastic net, lasso, Bayesian approach etc.

In one embodiment, the subject is European.

Subject Stratification

The degree of weight loss predicted by the method of the present invention may also be compared to one or more pre-determined thresholds. Using such thresholds, subjects may be stratified into categories which are indicative of the degree of predicted weight loss, e.g. low, medium, high and/or very high predicted degree of weight loss. The extent of the divergence from the thresholds is useful to determine which subjects would benefit most from certain interventions. In this way, dietary intervention and modification of lifestyle can be optimised, and realistic expectations of the weight loss to be achieved by the subject can be set.

In one embodiment, the categories include weight loss resistant subjects and weight loss sensitive subjects.

By the term “weight loss resistant” is meant a predicted degree of weight loss which is less than a predetermined value. Preferably “weight loss resistant” is defined as a subject having a weight loss percentage inferior to a predetermined value e.g. a subject predicted to lose less weight than the 10^{th}, 15^{th}, 20^{th} or 30^{th} percentile of the expected weight loss for the subject.

Preferably the degree of weight loss is represented by the number of BMI units lost, where BMI loss=((BMI1−BMI2)*100)/BMI1, wherein BMI1 is the body mass index of the subject before the dietary intervention and BMI2 is the predicted body mass index of the subject after the dietary intervention.

By the term “weight loss sensitive” is meant a predicted degree of weight loss of more than a predetermined value. Preferably “weight loss sensitive” is defined as a subject having a weight loss percentage superior to a predetermined threshold value. For example a subject predicted to lose more weight than the 85^{th}, 80^{th} or 75^{th} percentile of the expected weight loss.

The “expected weight loss” can be obtained from data of a population of subjects that have undergone the same dietary intervention as the one being tested.

In another embodiment, subjects may be stratified into categories “weight loss sensitive” or “weight loss resistant” which are indicative of the risk reduction of the subject for obesity or obesity-related disorders, e.g. low, medium, high and/or very high risk reduction. Low, medium and high risk reduction groups may be defined in terms of absolute weight loss, where the absolute weight loss relates to clinical criteria for obesity or a particular obesity-related disorder.

For example, if the aim is to reduce the risk for type 2 diabetes in an obese individual, “very high risk reduction” may be defined as those predicted to lose at least 10% body weight after the dietary intervention. This is in accordance with the criteria set out in Part II of the World Health Organ Tech Rep Ser. 2000; 894:i-xii, 1-253). Moreover every 1% reduction in body weight of an obese person leads to a fall in systolic and diastolic blood pressure, and fall in low-density lipoprotein cholesterol, hence reduces the risk of cardio-vascular disease and dyslipidaemia respectively.

Method for Selecting a Modification of Lifestyle of a Subject

In a further aspect, the present invention provides a method for modifying the lifestyle of a subject. The modification in lifestyle in the subject may be any change as described herein, e.g. a change in diet, more exercise, a different working and/or living environment etc.

Preferably the modification is a dietary intervention as described herein. More preferably the dietary intervention includes the administration of at least one diet product. The diet product preferably has not previously been consumed or was consumed in different amounts by the subject. The diet product may be as described herein. Modifying a lifestyle of the subject also includes indicating a need for the subject to change his/her lifestyle, e.g. prescribing more exercise or stopping smoking.

For example, if a subject is not predicted to lose weight on a low calorie diet, a modification may include more exercise in the subject's lifestyle.

Use of Diet Products

In one aspect, the present invention provides a diet product for use as part of a low calorie diet for weight loss. The diet product being administered to a subject that is predicted to attain a degree of weight loss by the methods described herein.

In another aspect, the present invention provides a diet product for use in treating obesity or an obesity-related disorder, wherein the diet product is administered to a subject that is predicted to attain a degree of weight loss by the methods described herein.

The obesity-related disorder may be selected from the group consisting of diabetes (e.g. type 2 diabetes), stroke, high cholesterol, cardiovascular disease, insulin resistance, coronary heart disease, metabolic syndrome, hypertension and fatty liver. In a further aspect, the present invention provides the use of a diet product in a low calorie diet for weight loss where the diet product is administered to a subject that is predicted to attain a degree of weight loss by the methods described herein.

Kits

In a further aspect, the present invention provides a kit for predicting the degree of weight loss attainable by applying one or more dietary interventions to the subject.

The kit comprises an antibody specific for gelsolin, and/or an antibody specific for apolipoprotein B-100, and/or an antibody specific for plasma kallikrein, and/or an antibody specific for protein Z-dependent protease inhibitor, and/or an antibody specific for plasma serine protease inhibitor. The kit preferably comprises at least two of said antibodies.

Preferably the kit comprises an antibody specific for gelsolin and an antibody specific for apolipoprotein B-100 and an antibody specific for plasma kallikrein and an antibody specific for protein Z-dependent protease inhibitor and an antibody specific for plasma serine protease inhibitor.

The term antibody includes antibody fragments. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. Furthermore, the antibodies and fragments thereof may be humanised antibodies. The skilled person will be aware of methods in the art to produce the antibodies required for the present kit.

Computer Program Product

The methods described herein may be implemented as a computer program running on general purpose hardware, such as one or more computer processors. In some embodiments, the functionality described herein may be implemented by a device such as a smartphone, a tablet terminal or a personal computer.

In one aspect, the present invention provides a computer program product comprising computer implementable instructions for causing a programmable computer to predict the degree of weight loss based on the levels of biomarkers as described herein.

In another aspect, the present invention provides a computer program product comprising computer implementable instructions for causing a device to predict the degree of weight loss given the levels of one or more biomarkers from the user, wherein the biomarkers are selected from gelsolin, apolipoprotein B-100, plasma kallikrein, protein Z-dependent protease inhibitor and plasma serine protease inhibitor.

Preferably the biomarker levels are fasting levels. The computer program product may also be given anthropometric measures and/or lifestyle characteristics from the user. As described herein, anthropometric measures include age, weight, height and body mass index and lifestyle characteristics include smoking status.

In a particularly preferred embodiment, the user inputs into the device levels of gelsolin, apolipoprotein B-100, plasma kallikrein, protein Z-dependent protease inhibitor and plasma serine protease inhibitor optionally along with age and body mass index. The device then processes this information and provides a prediction on the degree of weight loss attainable by the user from a dietary intervention.

The device may generally be a server on a network. However, any device may be used as long as it can process biomarker data and/or anthropometric and lifestyle data using a processor, a central processing unit (CPU) or the like. The device may, for example, be a smartphone, a tablet terminal or a personal computer and output information indicating the degree of weight loss attainable by the user.

Those skilled in the art will understand that they can freely combine all features of the present invention described herein, without departing from the scope of the invention as disclosed.

Various preferred features and embodiments of the present invention will now be described by way of non-limiting examples.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and E. M. Shevach and W. Strober, 1992 and periodic supplements, Current Protocols in Immunology, John Wiley & Sons, New York, N.Y. Each of these general texts is herein incorporated by reference.

### EXAMPLES

### Example 1—Predicting Women's Weight Loss after LCD Using a Combination of Blood Plasma Biomarkers and Anthropometric Measurements

Subjects were participants in the Diogenes study. This study is a pan-European, randomised and controlled dietary intervention study investigating the effects of dietary protein and glycaemic index on weight loss and weight maintenance in obese and overweight families in eight European centres (Larsen et al., Obesity reviews (2009), 11, 76-91).

The trajectory of weight loss was investigated in a cohort of overweight/obese individuals enrolled into an eight week LCD weight loss program (Larsen et al 2010).

The study consisted of 938 European individuals of which 782 finished the 8 week LCD program and 714 had all the required measurements with ranges admissible for a living subject. General characteristics for the individuals are shown in table 1.

TABLE 1
General characteristics of individuals
who followed the low calorie diet

| | Average (standard deviation) | |
|---|---|---|
| women percentage | 64 | (not applicable) |
| age | 41.5 | (6.3) |
| bmi before LCD | 34.6 | (4.9) |
| bmi after LCD | 30.8 | (4.4) |

Fasting blood was taken and plasma obtained from all the participants shortly before their adherence to an eight week low caloric dietary intervention. Fasting plasma samples for 294 women were available for which multiple biomarker′ levels were determined. Multiple anthropometric measures were also taken prior to this dietary intervention. A few relevant examples of these measures are: age, weight and height (from which the bmi—body mass index—is derived as weight/height²) and gender.

All the variables measured prior to the dietary intervention were evaluated for being separately and jointly predictors of bmi2 given bmi1. We evaluated multiple statistical models using freely available tools (R software) and retained following predictive model for men (based on the prediction quality using cross-validation):

bmi2ᵢ=c1*bmi1ᵢ+c2*ageᵢ−c3*gelsolinᵢ−c4*plasma kallikreinᵢ−c5*protein Z-dependent protease inhibitorᵢ+c6*apolipoprotein B-100ᵢ+c7*plasma serine protease inhibitorᵢ; (1)

where coefficients c1, c2, c3, c4, c5, c6, c7 are positive and their values depend on 1) the measurement units of all the variables in the model; and 2) provenance (ethnic background) of the considered subject.

The overall explanatory accuracy of the model in this study was determined to be 96% of the total variation (adjusted R2=0.96). In Table 2 we demonstrate the significance of all the coefficients of the predictive model for the average expected bmi2 (using Bayesian credible intervals at 99%).

TABLE 2
Coefficients when predicting average expected bmi2 with
regression as in (1) and Bayesian posterior probabilities
of corresponding coefficients to be greater than 0. Computations
use the model Bayesian regression model estimator proposed
by MacLehose et al. (P2 model).

| | | Bayesian posterior |
|---|---|---|
| | | probability for the |
| | | coefficient to be |
| | coefficient | greater than 0 |
| Gelsolin | c3 | >0.90 |
| Plasma kallikrein | c4 | >0.90 |
| Protein Z-dependent protease inhibitor | c5 | >0.95 |
| Apolipoprotein B-100 | c6 | >0.95 |
| Plasma serine protease inhibitor | c7 | >0.99 |

### Example 2: Stratification of Women According to Predicted Weight Loss and Success Thresholds

The term “weight loss resistant” is to be interpreted as being predicted to have a weight loss percentage inferior to a pre-determined threshold value. As an example “weight loss resistance” may be defined as predicted to lose less bmi units than the 30^{th} or 15^{th} percentile of the expected bmi loss (where bmi loss=(bmi1−bmi2)*100%/bmi1).

The term “weight loss sensitive” is to be interpreted as being predicted to have a weight loss percentage superior to a pre-determined threshold value. As an example “weight loss sensitivity” may be defined as predicted to lose more bmi units than the 70^{th} or 90^{th} percentile of the expected bmi loss.

The expected average median or other percentiles of the bmi loss can be obtained by a skilled person on a sample of subjects (from the population of interest) that has undergone a dietary intervention similar to the one to be used.

Receiver Operating Characteristic (ROC) curve is a “best-developed statistical tool for describing the performance of” diagnostic tests measured on continuous scale (see Pepe, M. S. (2003). The Statistical Evaluation of Medical Tests for Classification and Prediction, Oxford University Press, New York, page 66). ROC use is based on the dichotomization of the test outcome. In our case we define the group of “weight loss resistant” subjects and predict the probability of subject to be in this group prior to the dietary intervention. We consider two definitions of “weight loss resistant” corresponding to losing less than 10 or 8% of initial weight.

Numerical indices of the ROC curves are frequently used to summarize the curves. These summary measures are used as the basis for comparing ROC curves. The Area Under the ROC curve (AUC) is the most widely used summary measure. A perfect diagnostic test with the perfect ROC curve has the value AUC=1.0, while an uninformative test has AUC=0.5. Table 3 demonstrates biomarkers' ROC AUCs jointly and separately for predicting the probability of being “weight loss resistant”.

TABLE 3
Women biomarkers' ROC AUC in assessing quality of prediction for probability
to be assigned correctly to a group “weight loss resistant” for
women (depending on two different definitions of weight loss resistance).

| | ROC AUC when | ROC AUC when |
|---|---|---|
| | predicting probability to | predicting probability to |
| | be in the “weight loss | be in the “weight loss |
| | resistant” group i.e. | resistant” group i.e. |
| Biomarker | lose <10% of initial weight | lose <8% of initial weight |
| Gelsolin | 0.55 | 0.55 |
| Apolipoprotein B-100 | 0.55 | 0.69 |
| Plasma kallikrein | 0.52 | 0.56 |
| Plasma serine protease inhibitor | 0.52 | 0.62 |
| Protein Z-dependent protease | 0.52 | 0.60 |
| inhibitor | | |
| Jointly gelsolin and plasma | 0.61 | 0.67 |
| serine protease inhibitor | | |
| Jointly gelsolin, plasma serine | 0.63 | 0.67 |
| protease inhibitor and protein Z- | | |
| dependent protease inhibitor | | |
| Jointly gelsolin, plasma serine | 0.65 | 0.75 |
| protease inhibitor, protein Z- | | |
| dependent protease inhibitor and | | |
| apolipoprotein B-100 | | |
| All 5 markers jointly | 0.67 | 0.76 |

1. A method for predicting the degree of weight loss in a female subject attainable by applying one or more dietary interventions to a subject, said method comprising:
determining the level of one or more biomarkers in one or more samples obtained from the subject, wherein the biomarkers are selected from gelsolin, apolipoprotein B-100, plasma kallikrein, protein Z-dependent protease inhibitor and plasma serine protease inhibitor.

2. The method according to claim 1, wherein the method comprises determining the level of gelsolin in one or more samples.

3. The method according to claim 2, wherein the method further comprises determining the level of apolipoprotein B-100, plasma kallikrein, protein Z-dependent protease inhibitor or plasma serine protease inhibitor in one or more samples.

4. The method according to claim 3, wherein the method comprises determining the level of gelsolin, apolipoprotein B-100, plasma kallikrein, protein Z-dependent protease inhibitor and plasma serine protease inhibitor in one or more samples.

5. The method according to claim 4, wherein levels of each of gelsolin, apolipoprotein B-100, plasma kallikrein, protein Z-dependent protease inhibitor, and plasma serine protease inhibitor are determined, and decreased levels of apolipoprotein B-100 and plasma serine protease inhibitor and increased levels gelsolin, plasma kallikrein and protein Z-dependent protease inhibitor in the sample is indicative of a greater degree of weight loss in the subject.

6. The method according to claim 1, wherein the one or more samples are derived from blood.

7. The method according to claim 1, wherein the dietary intervention is a low calorie diet.

8. The method according to claim 7, wherein the low calorie diet comprises a calorie intake of about 600 to about 1200 kcal/day.

9. The method according to claim 7, wherein the low calorie diet comprises administration of at least one diet product.

10. The method of claim 7, wherein the low calorie diet has a duration of 6 to 12 weeks.

11. The method according to claim 1, wherein the method further comprises combining the level of the one or more biomarkers with one or more anthropometric measures and/or lifestyle characteristics of the subject.

12. The method according to claim 11, wherein the anthropometric measures include age and body mass index.

13. The method according to claim 1, wherein the degree of weight loss is represented by the body mass index that a subject is predicted to attain by applying a dietary intervention.

14. A method for optimizing one or more dietary interventions for a female subject comprising:
predicting the degree of weight loss attainable by the subject according to a method as defined in claim 1; and
applying the dietary intervention to the subject.

15. A method for predicting the body mass index that a female subject would be expected to attain from a dietary intervention (BMI2), wherein the method comprises:
a. determining the level of gelsolin, apolipoprotein B-100, plasma kallikrein, protein Z-dependent protease inhibitor and plasma serine protease inhibitor in one or more samples obtained from the subject; and
b. predicting BMI2 using formula (1):
bmi2ᵢ=c1*bmi1ᵢ+c2*ageᵢ−c3*gelsolinᵢ−c4*plasma kallikreinᵢ−c5*protein Z-dependent protease inhibitorᵢ+c6*apolipoprotein B-100ᵢ+c7*plasma serine protease inhibitorᵢ;
wherein BMI1 is the subject's body mass index before the dietary intervention and BMI2 is the subject's predicted body mass index after the dietary intervention; and wherein c1, c2, c3, c4, c5, c6 and c7 are positive integers.

16. A method for selecting a modification of lifestyle of a subject, the method comprising:
a. performing a method as described in claim 1; and
b. selecting a suitable modification in lifestyle based upon the degree of weight loss predicted in step (a).

17. The method according to claim 16, wherein the modification of lifestyle in the subject comprises a dietary intervention.

18. The method according to claim 17, wherein the dietary intervention as a low calorie diet.

19. A diet product for use as part of a low calorie diet for weight loss, wherein the diet product is administered to a female subject that is predicted to attain a degree of weight loss by the method of claim 1.

20. A diet product for use in treating obesity or an obesity-related disorder, wherein the diet product is administered to a female subject that is predicted to attain a degree of weight loss by the method of claim 1.

21. Use of a diet product in a low calorie diet for weight loss wherein the diet product is administered to a female subject that is predicted to attain a degree of weight loss by the method of claim 1.

22. A computer program product comprising computer implementable instructions for causing a programmable computer to perform the method of any of claim 1.

23. A computer program product comprising computer implementable instructions for causing a programmable computer to predict the degree of weight loss in a female subject given the levels of one or more biomarkers from the user, wherein the biomarkers are selected from gelsolin, apolipoprotein B-100, plasma kallikrein, protein Z-dependent protease inhibitor and plasma serine protease inhibitor.

24. The product according to claim 23, wherein the computer program product is further given anthropometric measures and/or lifestyle characteristics from the user.

25. The product according to claim 24, wherein anthropometric measures include age and body mass index.

26. A kit for predicting the degree of weight loss attainable by a female subject following a dietary intervention, wherein said kit comprises two or more of:
a. an antibody specific for gelsolin;
b. an antibody specific for apolipoprotein B-100;
c. an antibody specific for plasma kallikrein;
d. an antibody specific for protein Z-dependent protease inhibitor; and
e. an antibody specific for plasma serine protease inhibitor.

27. A kit according to claim 26 comprising
a. an antibody specific for gelsolin;
b. an antibody specific for apolipoprotein B-100;
c. an antibody specific for plasma kallikrein;
d. an antibody specific for protein Z-dependent protease inhibitor; and
e. an antibody specific for plasma serine protease inhibitor.
